(19) United States
(12) Patent Application Publication
(10) Pub. No.: US 2007/0225801 A1
(43) Pub. Date: Sep. 27, 2007

(54) VALVE INTRODUCERS AND METHODS FOR MAKING AND USING THEM

(76) Inventors: Michael J. Drews, Palo Alto, CA (US); Donnell W. Gurskis, Belmont, CA (US); Takashi Harry Ino, San Jose, CA (US); Oleg Svanidze, Laguna Hills, CA (US)

Correspondence Address: Vista IP Law Group LLP, 2040 MAIN STREET, 9TH FLOOR, IRVINE, CA, 92614, US

(21) Appl. No.: 11/685,192

(22) Filed: Mar. 12, 2007

Related U.S. Application Data
(60) Provisional application No. 60/781,065, filed on Mar. 10, 2006.

(51) Int. Cl. A61F 2/24

(52) U.S. Cl. 623/2.11; 623/2.38

(57) ABSTRACT

A valve introducer is provided for delivering a valve prosthesis into a biological annulus. During use, a gasket member is introduced into the biological annulus, and secured relative to the biological annulus. A distal end of a valve introducer is introduced into a passage communicating with the biological annulus, and disposed adjacent the gasket member. A valve prosthesis is advanced through the valve introducer towards the gasket member, and secured to the gasket member. In one embodiment, the valve introducer and valve prosthesis have corresponding shapes, requiring the valve prosthesis to be oriented to properly align the valve prosthesis before advancing the valve prosthesis into the valve introducer. Optionally, the valve introducer is compressed or otherwise manipulated to reduce a profile of the distal end before introduction into the passage, which may facilitate introducing the valve introducer through the passage, e.g., past the sino-tubular junction.

### RELATED APPLICATION DATA

This application claims benefit of co-pending provisional application Ser. No. 60/781,065, filed Mar. 10, 2006, the entire disclosure of which is expressly incorporated by reference herein.

### FIELD OF THE INVENTION

The present invention relates generally to heart valves that may be implanted within a patient. More particularly, the present invention relates to valve introducers that may be used to deliver a prosthetic valve into a biological annulus, e.g., past a sino-tubular junction into a sinus above an aortic valve site, and to methods for using such valve introducers.

### BACKGROUND

Prosthetic heart valves can replace defective human valves in patients. For example, one piece valves have been suggested that include sewing rings or suture cuffs that are attached to and extend around the outer circumference of a prosthetic valve. In addition, multiple component valves have also been suggested that include a sewing ring that is separate from a valve component. The sewing rings of either type of prosthetic valve can be tedious and time consuming to secure within a target site, i.e., within an annulus of a heart where a natural heart valve has been removed.

For example, to implant a sewing ring within a biological annulus of a heart, between twelve and twenty sutures may be secured initially to tissue surrounding the biological annulus. The sewing ring and/or the entire prosthetic valve may then be advanced or “parachuted” down the sutures into the biological annulus. Knots may then be tied with the sutures to secure the sewing ring within the biological annulus, whereupon the sutures may be cut. Consequently, this procedure can be very complicated, requiring management and manipulation of many sutures. The complexity of the procedure also provides a greater opportunity for mistakes and requires a patient to be on cardiopulmonary bypass for a lengthy period of time.

Because the biological annulus of the heart may not match the circular cross-section of the sewing ring and/or prosthetic valve, the prosthetic valve may not fit optimally within the biological annulus. As a result, natural blood hemodynamics through and around the valve may be impaired, resulting in clotting, possible emboli production, and eventual calcification of the valve structure.

To address this concern, flexible sewing rings have been suggested for use with multiple component valves. The sewing ring may be implanted within the biological annulus, e.g., using the procedure described above, i.e., parachuted down an arrangement of sutures. The sewing ring may conform at least partially to the anatomy of the biological annulus. Alternatively, instead of using sutures, it has also been suggested to drive staples through the sewing ring into the surrounding tissue to secure the sewing ring.

Once the sewing ring is secured within the biological annulus, a valve prosthesis, e.g., a bioprosthetic or mechanical valve may be introduced and secured to the sewing ring. Often because of limited access to the biological annulus, e.g., through an aortic access opening, it may be difficult to introduce the valve prosthesis, e.g., past the sino-tubular junction into the sinus cavity above the native valve site. In addition, the flexible sewing ring may be slightly distorted and/or may have features that help with sealing and seating of the valve that may be obscured and/or may prevent the valve prosthesis from reaching a desired target area or landing zone of the sewing ring.

Accordingly, apparatus and methods for facilitating access to an implantation site and/or to aid with the implantation itself, e.g., for delivering a prosthetic valve into a sinus cavity above a biological annulus, and/or for guiding the prosthetic valve within features on the sewing ring would be useful.

### SUMMARY

The present invention is directed to apparatus and methods for implanting heart valves within a biological annulus within a patient, and, more particularly, to valve introducers for delivering one or more components of a heart valve assembly into a biological annulus, e.g., for delivering a prosthetic heart valve into a biological annulus. Such apparatus and methods may facilitate access to an implantation site, may aid with the implantation itself, may guide a prosthetic valve within a sewing ring or collar of the gasket, and/or may guide the prosthetic valve within features on the gasket used to help seal and/or seat the prosthetic valve.

In accordance with one embodiment, a valve introducer is provided that includes a tubular body having a proximal end for receiving a prosthetic valve therein, and a distal end sized for introduction into a biological annulus and/or onto a previously placed gasket or other annular member, e.g., for guiding the prosthetic valve therein. In one embodiment, at least a portion of the tubular body may have a cross-section similar to the prosthetic valve, e.g., a multiple lobed or sided shape, thereby substantially maintaining the prosthetic valve in a desired angular orientation during introduction through the valve introducer.

Optionally, the valve introducer may be tapered, e.g., such that the distal end is smaller than the proximal end. In addition or alternatively, the valve introducer may be movable, e.g., for reducing a profile of the distal end. For example, the distal end may include a plurality of petals that may be manipulated to reduce the profile of the distal end, e.g., to facilitate insertion into a biological annulus and/or onto a previously placed gasket. Alternatively, the valve introducer may include a pivot point, e.g., adjacent the proximal end, allowing the valve introducer to be compressed at a location below the pivot point to reduce the profile of the distal end. In addition or alternatively, the tubular body may include longitudinal pleats that allow the valve introducer to be circumferentially compressed and/or expanded, e.g., to accommodate patient anatomy with minimized distortion to the valve introducer and/or to accommodate introduction of the prosthetic valve during implantation.

Optionally, the distal end may have petals or other tip features, e.g., that may be curved inwardly, for example, to fit within an inner diameter of a biological annulus, a sewing ring, and/or to fit within features on a gasket or other annular member used to help seal and/or seat the prosthetic valve.

In an exemplary embodiment, the valve introducer may be a flat sheet of material shaped such that the sheet may be folded or rolled into a tubular body. Opposing edges of the flat sheet may include cooperating connectors, e.g., one or more mating tabs and slots, that may secure the tubular body after folding or rolling the flat sheet and/or the opposing edges may be removably or substantially permanently bonded together. The valve introducer may be formed from a thin sheet of material, e.g., a plastic, such as mylar, that may be laser, die, or otherwise cut into the desired shape and/or to include any desired features, e.g., the cooperating connectors. The sheet may include grooves, thinned regions, and the like to provide seams for folding in a desired manner, e.g., to bias the sheet to be folded into a predetermined multiple sided shape.

In another exemplary embodiment, the valve introducer may be fabricated from a flat polymer sheet material formed into a tubular body and/or shape, e.g., created by a thermo-forming process, such as vacuum forming, deep-drawn forming, or any other thermal method of creating a three-dimensional shape from sheet material. In such processes, the sheet may take the form dictated by the mold used, which may be configured based upon the desired final configuration.

In yet another exemplary embodiment, the valve introducer may be fabricated from a heat-shrinkable tubular polymer, e.g., polytetrafluoroethylene (PTFE), TetraFluorEthylene-Perfluorpropylene (FEP), Polyethylene terephthalate (PET), and the like, which may be heated over a mandrel or otherwise formed to create the desired shape.

In still another exemplary embodiment, the valve introducer may be fabricated from sheet, tubular, or capped tubular polymer material, e.g., using a blow-molding process capable of creating elongated tubular shapes, e.g., corresponding to the shape of the cavity in the mold used.

In another exemplary embodiment, the valve introducer may be fabricated by placing a capped tubular material over a form and applying a vacuum, e.g., from within the capped tubular material, to draw the tubular material to conform to the shape of the form. Heat may then be applied, e.g., to reflow, heat set, and the like, to enable the polymer to take a permanent set shape of the form used. The form may have longitudinal ridges, e.g., along a longitudinal axis of the valve introducer, that may create pleat-like features in the valve introducer, e.g., to allow for a desired amount of radial expandability. The pleat-like features may also allow the valve introducer to reduce in size by locally compressing the pleats and/or may minimize the overall distortion on the valve introducer, which may ensure placing the prosthetic valve within the valve introducer.

For any of the aforementioned forming processes, additional desired features in the valve introducer may be created, e.g., by die cutting, razor blade type-cutting, laser cutting, or any other cutting method known in the art.

In accordance with another embodiment, a system or kit is provided for implanting a heart valve assembly within a biological annulus. The heart valve assembly may include an annular prosthesis implantable within a biological annulus, a prosthetic valve, e.g., including a mechanical or bioprosthetic heart valve, and a valve introducer. The valve introducer may include any of the features described elsewhere herein.

In an exemplary embodiment, the annular prosthesis may include a plurality of elongate rails or other elements extending therefrom, and the valve introducer may include a plurality of holes adjacent its distal end that may received respective elongate elements therethrough, e.g., to angularly align the valve introducer and the annular prosthesis. Optionally, the annular prosthesis may include a collar or other seat extending upwardly therefrom, and the distal end of the valve introducer may be configured to be received in or otherwise engaged with the collar, e.g., to facilitate introduction of the prosthetic valve through the valve introducer into the collar or seat.

In accordance with yet another embodiment, a method is provided for assembling a valve introducer from a flat sheet. The flat sheet may be rolled or folded into a tubular body, e.g., having a multiple sided shape corresponding generally to a shape of a prosthetic valve. Optionally, the flat sheet may include one or more connectors along opposing edges, and the one or more connectors may be secured together to secure the tubular body. In one embodiment, the one or more connectors may include a pivot point that allows a distal end of the tubular body to be compressed inwardly to reduce a profile of the distal end.

In accordance with still another embodiment, a method is provided for implanting a prosthetic heart valve assembly to replace a natural or prosthetic heart valve implanted within a biological annulus, e.g., into an aortic valve site below a sinus cavity. An annular member may be introduced into the biological annulus, e.g., to direct tissue surrounding the biological annulus outwardly and/or to at least partially dilate the biological annulus. A flexible sewing cuff or skirt may extend around the annular member that may receive one or more connectors, e.g., sutures, clips, and the like, to secure the annular member within the biological annulus.

A distal end of a valve introducer may be introduced into a passage communicating with the sinus cavity. Optionally, the distal end may be compressed or otherwise manipulated to reduce a profile of the distal end before or during introduction into the passage. The distal end may be positioned within the sinus cavity against or adjacent the annular member. Optionally, the valve introducer may include one or more holes, e.g., adjacent the distal end, that may receive or otherwise accommodate elongate rails or other elements extending from that the annular member. The elongate elements may be directed through the holes into the interior of the valve introducer, which may facilitate angularly orienting the valve introducer relative to the annular member and/or the biological annulus.

A prosthetic valve, e.g., a mechanical or bioprosthetic valve prosthesis, may be advanced into a proximal end of the valve introducer, and advanced therethrough into the biological annulus, e.g., into the sinus cavity above an aortic valve site. Optionally, the valve introducer may maintain the prosthetic valve in a desired angular orientation as the prosthetic valve is advanced through the valve introducer, thereby aligning the prosthetic valve with the annular member. In addition, if elongate elements extend from the annular member through the valve introducer, the elongate elements may also be used to guide the prosthetic valve towards the annular member and/or to secure the prosthetic valve relative to the annular member.

Optionally, one or more connection elements, e.g., barbs, detents, tabs, knots, or other connectors, may be provided on the elongate elements adjacent the annular member. The connection elements may be received through the holes in the valve introducer to temporarily retain the valve introducer against or otherwise adjacent the annular member, e.g., to minimize the need for further manipulation or stabilization by the user during introduction of the prosthetic valve.

The prosthetic valve may then be secured relative to the annular member, e.g., using one or more connectors on the prosthetic valve and/or the annular member. For example, the annular member may include a collar, and the prosthetic valve may be secured within or against the collar. The valve introducer may then be removed.

Optionally, one or more perforations, score lines, weakened regions, seams, and/or lapped edges may be provided along the length of the valve introducer. Such features may provide a controllable path, allowing a user to easily separate the valve introducer for removal. Alternatively, mono-filament suture, multi-filament suture, cable, or wire may be provided along the length of the valve introducer, e.g., as a single ended member or as a loop, that may be used to capture a portion of the valve introducer and aid in separating the valve introducer, e.g., similar to a rip-cord or tear strip. The valve introducer may or may not include a weakened path to facilitate the rip-cord to function. For example, a rip cord may easily tear through PTFE shrink tubing with only notch defect and may not require further weakening along the path.

Other aspects and features of the present invention will become apparent from consideration of the following description taken in conjunction with the accompanying drawings.

### BRIEF DESCRIPTION OF THE DRAWINGS

The drawings illustrate exemplary embodiments of the invention, in which:

FIG. 1 is a perspective view of a valve introducer inserted into a biological annulus.

FIG. 2 is a front view of the valve introducer of FIG. 1, having a valve frame disposed therein.

FIG. 3 is a top view of the valve introducer and valve frame of FIG. 2.

FIG. 4 is a front view of the valve introducer and valve frame of FIG. 2 placed adjacent a gasket member.

FIG. 5 is a perspective view of the valve introducer, valve frame, and gasket member of FIG. 4.

FIG. 6 is a plan view of a flat sheet cut into a shape to provide a valve introducer.

FIG. 7 is a plan view of another flat sheet cut into a shape to provide a tapered valve introducer.

FIGS. 8A and 8B are perspective views of another embodiment of a valve introducer movable between a relaxed configuration and a radially compressed configuration, respectively.

FIGS. 9A and 9B are front views of another embodiment of a valve introducer including a pivot point for reducing a profile of a distal end of the valve introducer.

FIGS. 10A and 10B are perspective views of a patient's body, showing a method for introducing a valve introducer into a passage communicating with a native valve annulus within which a gasket member has been secured.

FIGS. 11A-11C are perspective views of the body of FIGS. 10A and 10B, showing a prosthetic valve carried by a valve holder and being introduced through the valve introducer into the passage communicating with the native valve annulus.

FIGS. 12A-12C are perspective, side, and bottom views of another embodiment of a valve introducer.

FIGS. 13A-13D are perspective views of alternative embodiments of valve introducers including features for separating the valve introducers, e.g., to facilitate removal after being used to introduce a prosthetic valve therethrough.

FIGS. 14A-14C are perspective, side, and bottom views of yet another embodiment of a valve introducer, including longitudinal pleats.

FIGS. 15A-15D show a method for using the valve introducer of FIGS. 12A-12C to facilitate introduction and/or implantation of a heart valve assembly including an annular prosthesis and a prosthetic valve.

### DETAILED DESCRIPTION OF THE EXEMPLARY EMBODIMENTS

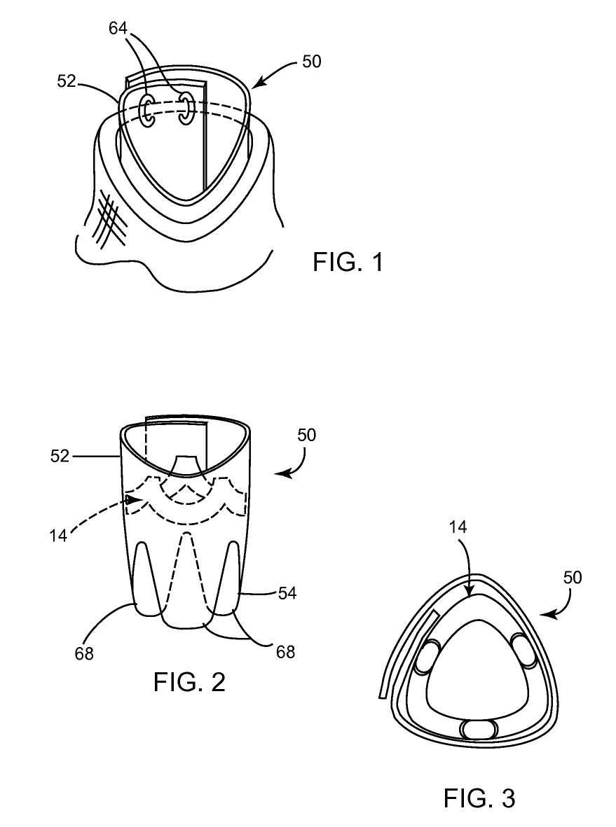

Turning to the drawings, FIGS. 1-3 show an exemplary embodiment of a valve introducer 50 that generally includes a tubular body having a proximal end 52 for receiving a prosthetic valve therein, and a distal end 54. In one embodiment, at least a portion of the valve introducer 50 may have a cross-section similar to a valve prosthesis 14 (shown in FIGS. 2 and 3), e.g., a multiple lobed or sided shape. As shown in FIGS. 1-3, the valve introducer 50 may include three sides defining three lobes or apices, which may correspond to the number of commissures in an aortic valve annulus. It will be appreciated that other numbers of sides may also be provided, e.g., four, five, or more, if desired. The sides may be curved, e.g., externally convex, thereby providing concave inner surfaces for facilitating guiding the valve prosthesis 14 into and through the valve introducer 50. In addition, the shape of the valve introducer 50 may allow the valve prosthesis 14 to be introduced into the proximal end 52 in a desired angular orientation and/or substantially maintain the valve prosthesis 14 in the desired angular orientation during introduction through the valve introducer 50, as described further below.

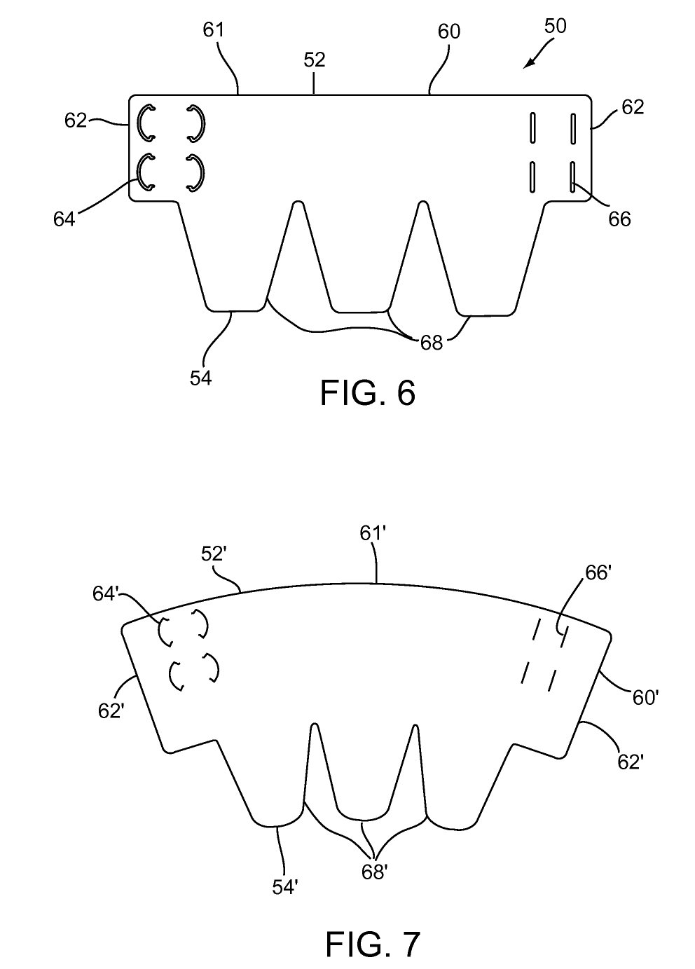

Turning to FIGS. 6-7, in an exemplary embodiment, the valve introducer 50 may be formed from a flat sheet 60 of material shaped such that the sheet 60 may be folded and/or rolled into the desired shape of the valve introducer 50. The sheet 60 may include an upper edge 61 defining the proximal end 52, a plurality of petals 68 defining the distal end 54, and opposing side edges 62. The opposing side edges 62 may include one or more cooperating connectors, e.g., tabs 64 and slots 66 as shown, that may secure the opposing edges 62 adjacent and/or against one another after folding and/or rolling the sheet 60. Optionally, the sheet 60 may include grooves, scoring lines, thinned regions, and the like (not shown) to provide seams for folding in a desired manner, e.g., to bias the sheet 60 to be folded into a predetermined multiple sided shape. For example, vertical grooves (not shown) may be provided that extend from between adjacent pairs of petals 68 to the upper edge 61 to enhance the sheet 60 bending between the petals 68.

During assembly, the sheet 60 may be folded and/or rolled, and then the tabs 64 may be inserted into respective slots 66, thereby securing the valve introducer 50 in the tubular shape. The tabs 64 may be removable from the slots 66 if it is desired to disassemble or otherwise at least partially separate the valve introducer 50 after use, e.g., to remove the valve introducer 50 from around a heart valve assembly (not shown), as described elsewhere herein. Alternatively, the tabs 64 may include barbs or other features that allow the tabs 64 to be inserted into the slots 66, but prevent subsequent removal. In addition or alternatively, the opposing edges 62 may be attached to one another, e.g., using an adhesive, sonic welding, fusing, melting, and the like.

The valve introducer 50 may be formed from a thin sheet of material, e.g., a plastic, such as mylar, polytetrafluoroethylene (PTFE), TetraFluorEthylene-Perfluorpropylene (FEP), Polyethylene terephthalate (PET), and the like. The sheet may have a thickness between about 0.0005-0.1 inch (0.125-2.5 mm), 0.001-0.02 inch (0.025-0.5 mm), or 0.002-0.005 inch (0.05-0.125 mm). The material may be sufficiently rigid to support the valve introducer 50 after assembly, yet be sufficiently flexible to allow the petals 68 and/or other components of the valve introducer 50 to be deflected, as described elsewhere herein. In addition, transparent materials may facilitate monitoring tissue surrounding the valve introducer 50 during use and/or monitoring a prosthetic valve being introduced through the valve introducer 50, although alternatively opaque materials may be used. The sheet 60 may be laser cut, die cut, or otherwise formed into the desired shape and/or to include any desired features, e.g., the cooperating connectors 64, 66.

Alternatively, the valve introducer 50 may be formed as a continuous walled tubular body, e.g., by extruding, injection molding, molding over a heated mandrel, thermo-forming processes, such as vacuum forming, deep-drawn forming, and the like, thereby eliminating the need for the connectors.

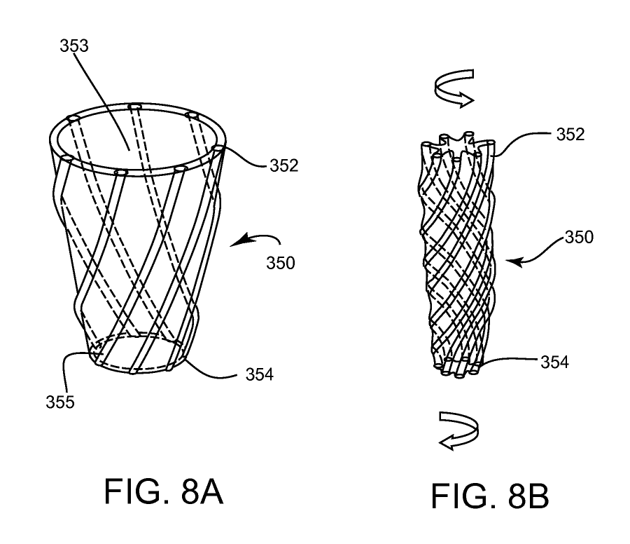

In a further alternative, the valve introducer 50 may be formed from a coiled or braided structure, e.g., interwoven strips of plastic or other material (not shown), that may be self-supporting, yet may be manipulated to change the shape and/or configuration of the valve introducer 50, e.g., to radially compress and/or expand the valve introducer 50. For example, FIGS. 8A and 8B show an exemplary embodiment of a valve introducer 350 that includes a tubular body including proximal and distal ends 352, 354, similar to the other embodiments described herein. Optionally, the valve introducer 350 may include one or more petals, holes, and/or other features (not shown), similar to other embodiments herein. As shown, the tubular body includes a thin walled material 353 supported by a plurality of helical supports 355. For example, the thin walled material 353 may be fabric, and the supports 355 may be formed from solid or hollow rods or tubes embedded within or attached to the fabric. As shown in FIG. 8B, the ends 352, 354 of the valve introducer 350 may be rotated in opposite directions to wind and/or radially compress the valve introducer 350. The valve introducer 350 may then be introduced into biological annulus, and then released, whereupon the valve introducer 350 may resiliently return to shape shown in FIG. 8A, which may dilate tissue surrounding the biological annulus and/or stabilize and/or secure the valve introducer 350 relative to the biological annulus.

In another embodiment, the valve introducer 50 may be formed from cloth or fabric, e.g., including ribs or other reinforcement elements woven into or attached to the fabric. The reinforcement elements may allow the fabric to be deflected, e.g., to compress the valve introducer 50, yet may be sufficiently resilient to bias the valve introducer 50 to return to a larger, relaxed configuration. Optionally, the valve introducer 50 may be formed from other materials, e.g., metal, such as stainless steel, plastic, or composite materials. Thus, the valve introducer 50 may be a single use device, or may be reusable, e.g., after resterilization.

Optionally, if the valve introducer 50 is formed as a continuous walled tubular body, the valve introducer 50 may include one or more seams, e.g., perforations, weakened regions, and/or rip cords or other features to allow the valve introducer 50 to be torn apart from the tubular shape, as described elsewhere herein.

Optionally, as shown in FIG. 2, the valve introducer 50 may be tapered, e.g., such that the distal end 54 is smaller than the proximal end 52. For example, the proximal end 52 may be relatively large compared to a prosthetic valve to be introduced through the valve introducer 50, e.g., to facilitate initial introduction into the proximal end 52. In addition or alternatively, the distal end 54 may be formed to have a smaller cross-section than the proximal end 52. For example, the petals 68 may be bent slightly inwardly to provide a tapered distal tip. To facilitate such bending, grooves, scoring, and the like may be provided across the base of the petals 68. Thus, the petals 68 may be biased into a tapered shape, yet be compressible or otherwise deflectable from the tapered shape.

As shown in FIG. 7, in an alternative embodiment, a sheet 60′ may be provided that has an arcuate shape, e.g., including a curved upper edge 61′ and nonparallel opposing edges 62.′ Upon forming the sheet 60′ into a valve introducer (e.g., by folding and/or rolling the sheet 60′ and securing the connectors 64,′ 66′), the resulting valve introducer (not shown) may have a frustoconical shape, e.g., with the distal end 54′ having a smaller cross-section than the proximal end 52.′ The valve introducer 50 may have a diameter between about 0.5 to two inches (12.5-50 mm), or about one to one and a half inches (25-37.5 mm).

In addition or alternatively, at least a portion of the valve introducer 50 may be movable for reducing a profile of the distal end 54. For example, with reference to FIG. 6, one or more of the petals 68 on the distal end 54 may be manipulated to reduce the profile of the distal end 54. For example, after assembling the valve introducer 50 from the sheet 60, one or more of the petals 68 may be directed inwardly to reduce the profile of the distal end 54, e.g., to facilitate insertion into a biological annulus (not shown). The petals 68 may be sufficiently resilient to be biased to return outwardly to their original shape upon release. For example, after inserting the distal end 52 through a biological annulus with the petal(s) 68 compressed, the petal(s) 68 may be released to at least partially dilate and/or direct tissue around the biological annulus outwardly. Thus, the petals 68 may facilitate access and/or visual monitoring through a relatively narrow biological annulus.

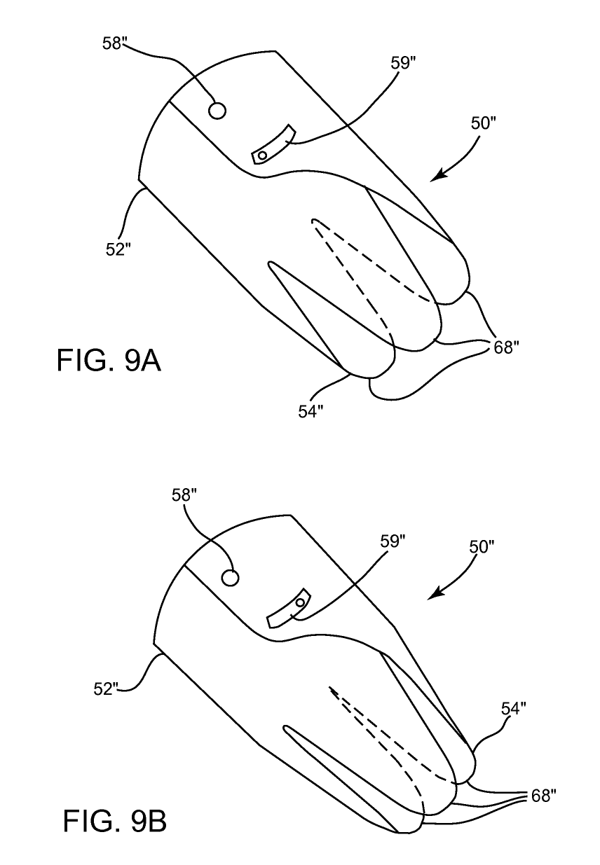

Alternatively, turning to FIGS. 9A and 9B, another embodiment of a valve introducer 50″ is shown that includes a pivot point 58,″ e.g., adjacent the proximal end 52.″ As shown, the valve introducer 50″ includes an upper set of connectors 58,″ e.g., a tab and slot similar to the embodiments of FIGS. 6 and 7, that substantially fix the proximal end 52.″ In addition, the valve introducer 50″ includes a lower set of connectors 59″ including a larger slot allowing a tab to be slidably received therein. If a radially inward force is applied to the valve introducer 50″ below the pivot point 58,″ the valve introducer 50″ may adopt a more conical shape, tapering towards the distal end 54.″ The valve introducer 50″ may be sufficiently resilient that, upon release of the inward force, the valve introducer 50″ may be biased to return outwardly to its original shape. Thus, the valve introducer 50″ may be compressed below the pivot point 58″ to reduce the profile of the distal end 54,″ which may facilitate introducing the valve introducer 50″ into a biological annulus (not shown). When released, the valve introducer 50″ may expand to contact tissue surrounding the biological annulus, which may dilate the surrounding tissue and/or frictionally stabilize the valve introducer 50″ relative to the biological annulus.

Optionally, the proximal end of any of embodiments of valve introducers described herein may include guiding elements. For example, ridges or tabs (not shown) may be formed or otherwise provided on the proximal end 52 of valve introducer 50 to provide guiding elements. The tabs may be bent or otherwise directed radially outwardly, e.g., similar to the petals 68 described above. The guiding elements may facilitate guiding a prosthetic valve (not shown) into the proximal end 52.

Optionally, before or after assembling the valve introducer 50, the valve introducer (or sheet) may be sterilized. A shape of the valve introducer 50 may be set before, during, or after sterilization, e.g., by heating or otherwise treating the material of the valve introducer 50.

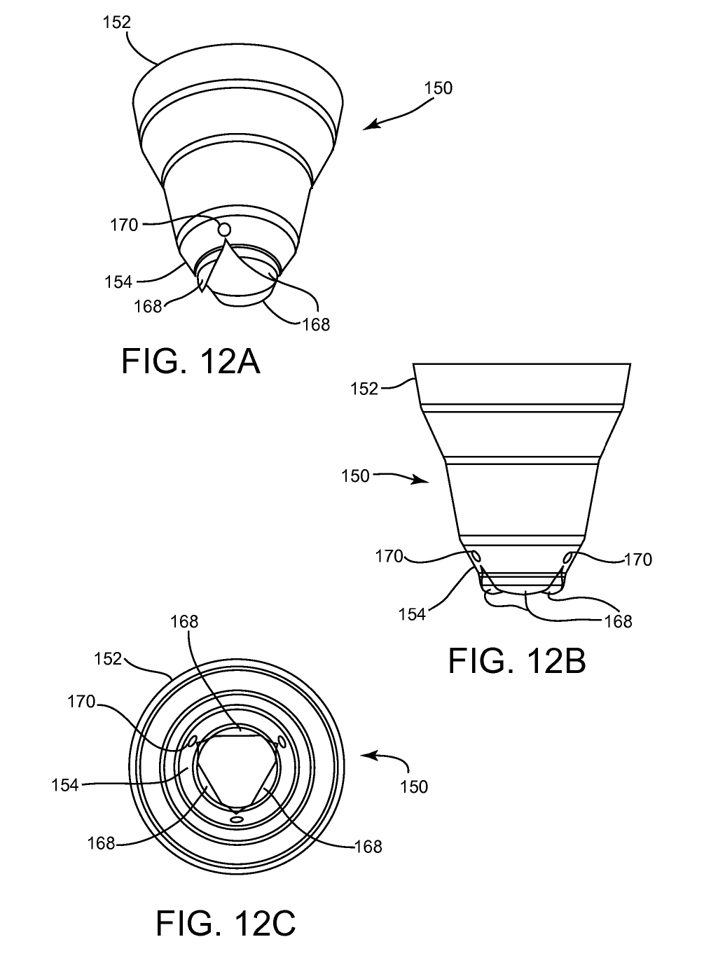

Turning to FIGS. 12A-12C, another embodiment of a valve introducer 150 is shown that includes a tubular body including a substantially circular proximal end 152 and a multi-lobular distal end 154. The tubular body may be formed by any of the materials and methods described elsewhere herein. For example, a flat polymer sheet may be formed into the tubular body by a thermo-forming process, e.g., vacuum forming, deep-drawn forming, or any other thermal method, which may create a three-dimensional shape from a sheet material. The mold may have a predetermined shape, such as that corresponding to the shape shown in FIGS. 12A-12C.

Alternatively, the tubular body may be formed from a heat-shrinkable tubular polymer, which may be heated over a mandrel or form to create the desired shape. In yet another alternative, the tubular body may be fabricated from a sheet, a tubular structure, or a capped tubular structure, e.g., using a blow-molding process. In still another alternative, the tubular body may be fabricated by placing a capped tubular material over a form and applying a vacuum from within the capped tubular material. The vacuum may draw the tubular material to conform to the shape of the form and then heat may be applied to reflow and/or heat set the shape of the form substantially permanently into the tubular body. In another embodiment, the valve introducer 150 may be formed from a sheet having overlapping longitudinal edges (not shown). Optionally, the overlapping edges may include one or more connectors, e.g., one or more tabs and slots (not shown), similar to previous embodiments. The overlapping edges may be biased to overlap one another, yet be sufficiently flexible to allow the edges to be at least partially separated, e.g., to facilitate removal of the valve introducer 150, as described elsewhere herein.

With continued reference to FIGS. 12A-12C, the valve introducer 150 may include one or more features, e.g., at or adjacent the distal end 154. For example, the distal end 154 may include a plurality of petals 168 spaced apart around the circumference, e.g., three petals as shown. In addition, a plurality of holes 170 may also be provided adjacent the distal end 154, e.g., located between adjacent petals 168, as shown. These or any other desired features may be created when the tubular body is formed, e.g., during a molding process, or may be formed thereafter, e.g., by die cutting, razor blade type-cutting, laser cutting, and the like.

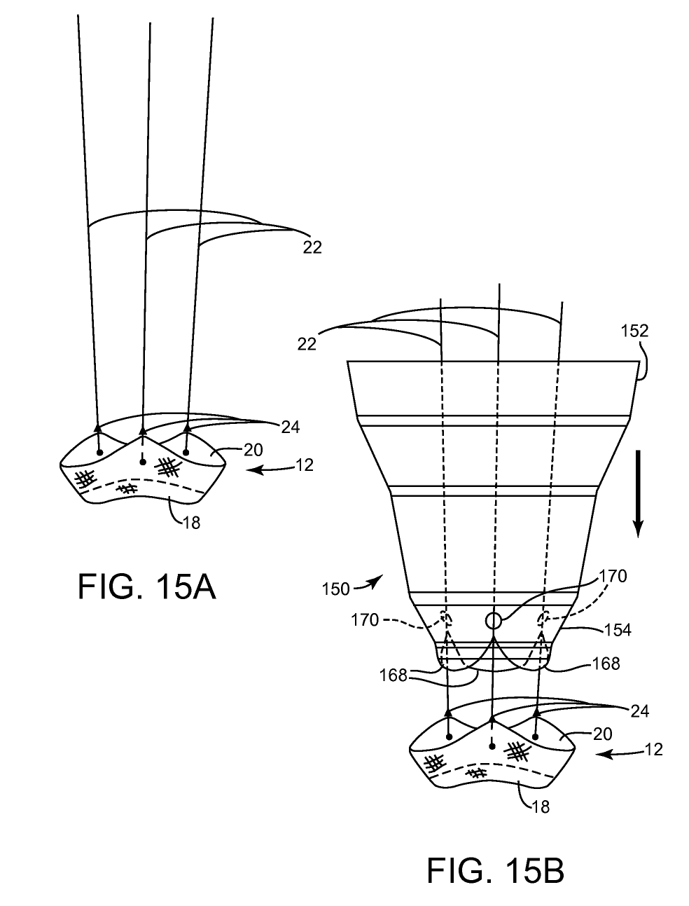

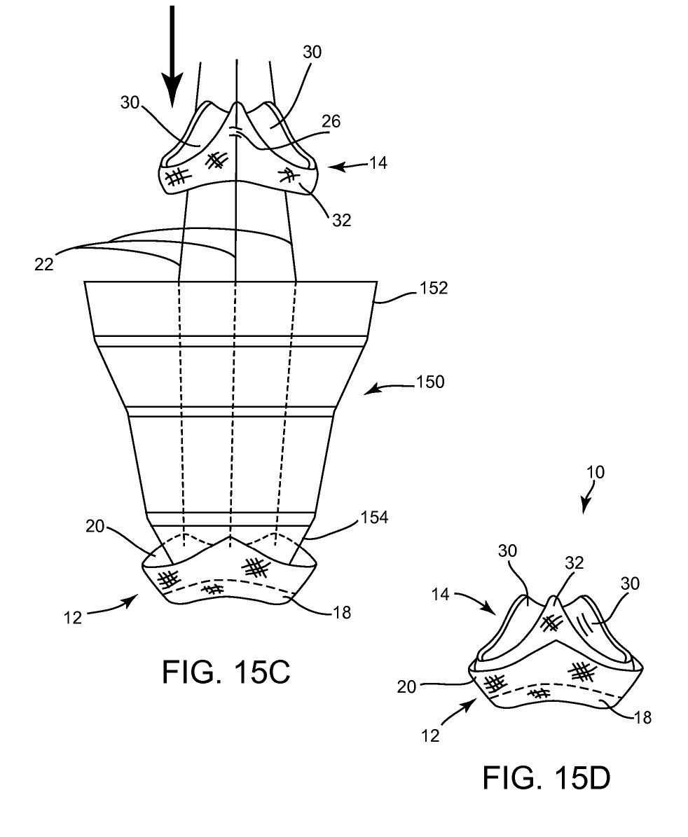

With additional reference to FIG. 15B, the holes 170 may be sized to receive respective guide rails or other elongate elements 22 extending from a gasket member 12, which may be constructed as described elsewhere herein and in the applications incorporated by reference herein. Optionally, the guide rails 22 may include detents or other connectors 24 adjacent the gasket member 12, which may be received through the holes 170 when the valve introducer 150 is directed towards the gasket member 12. Once the connectors 24 are received through the holes 170, the connectors 24 may prevent substantial proximal movement of the valve introducer 150 away from the gasket member 12, thereby securing the distal end 154 against, adjacent or otherwise relative to the gasket member 12. As shown, the configuration of the holes 170 and connectors 24 may secure the distal end 154 of the valve introducer 150 against and/or within the sewing cuff 20 of the gasket member 12. Alternatively, if the gasket member 12 includes a collar or other seat (not shown) extending upwardly from the annular ring 18 and/or sewing cuff 20, the petals 168 and/or the entire distal end 154 of the valve introducer 150 may be received within the collar or seat.

Optionally, the valve introducer 150 may include weakened regions, e.g., perforation, thinned regions, and the like (not shown) extending from the holes 170 to the distal end 154 between the adjacent petals 168. Alternatively, the holes 170 may be located sufficiently close to the distal end 154 to provide a region that may tear preferentially. Thus, with the guide rails 24 received in the holes 170, the valve introducer 150 may be pulled proximally, causing the valve introducer material adjacent the holes 170 to tear or otherwise sever to allow the valve introducer 150 to be removed from the gasket member 12 and/or guide rails 22.

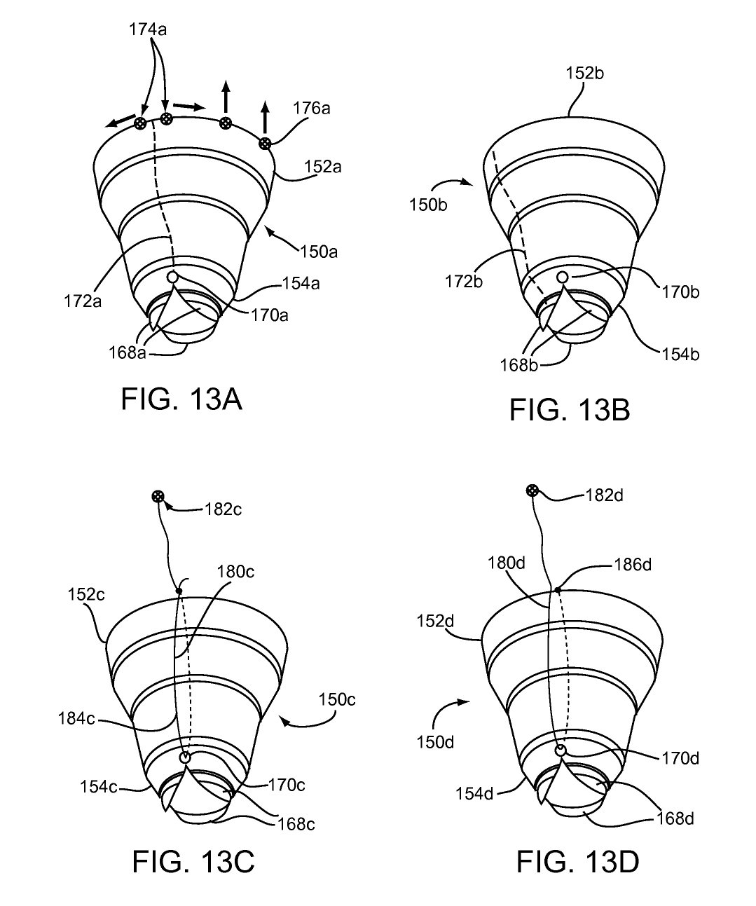

If desired, the valve introducer 150 may include one or more features that allow the valve introducer 150 to be torn or otherwise separated from a tubular body into one or more sheets. For example, turning to FIG. 13A, the valve introducer 150a is shown having a perforated or weakened region or seam 172a that extends between the proximal and distal ends 152a, 154a. If desired, one or more pull tabs 174a may be provided on the proximal end 154a, e.g., one on either side of the weakened seam 172a. For example, the pull tabs 174a may be pulled away from one another to cause the weakened seam 172a to tear from the proximal end 152a to the distal end 154a. Optionally, the valve introducer 150a may include one or more pull tabs 176a, handles, or other features (not shown) to facilitate holding and/or removing the valve introducer 150a after use.

Turning to FIG. 13B, another embodiment of a valve introducer 150b is shown that includes a weakened region or seam 172b that extends from the proximal end 152b through one of the petals 168b to the distal end 154b. Optionally, the valve introducer 150b may include one or more pull tabs (not shown), similar to the previous embodiment. It will be appreciated that multiple weakened regions or seams (not shown) may be provided in any of these embodiments, if desired to separate the valve introducer 150 into multiple pieces after use.

Turning to FIG. 13C, still another embodiment of a valve introducer 150c is shown that includes a tubular body including proximal and distal ends 152c, 154c, similar to the previous embodiments. As shown, the valve introducer 150c also includes a plurality of petals 168c and holes 170c. Unlike the previous embodiments, the valve introducer 150c includes a rip cord 180c that may be used to tear a seam from the distal end 154c to the proximal end 152c. The rip cord 180c includes a pull tab 182c on one end and a loop 184c that extends through one of the holes 170c to the proximal end 152c. Thus, when the pull tab 182c is pulled proximally relative to the valve introducer 150c, the loop 184c may tear proximally through the tubular body from the hole 170c to the proximal end 152c. Optionally, the valve introducer 150c may include a weakened region or seam (not shown) extending proximally from the hole 170c to facilitate preferential tearing of the valve introducer 150c.

Turning to FIG. 13D, yet another embodiment of a valve introducer 150d is shown that includes a tubular body including proximal and distal ends 152d, 154d, and may include a plurality of petals 168dand holes 170d, similar to the previous embodiments. As shown, the valve introducer 150d also includes a rip cord 180d including a pull tab 182d, similar to the previous embodiment. Unlike the previous embodiment, the rip cord 180d includes a second end 185d that is fixed to the proximal end 152d such that the rip cord 180d extends distally within the valve introducer 150d, through one of the holes 170d, and proximally along the exterior of the valve introducer 150d to the pull tab 180d. Thus, when the pull tab 180d is directed proximally relative to the valve introducer 150d, the rip cord 180d may tear through the tubular body from the hole 170d to the proximal end 152d of the valve introducer 150d. Optionally, the valve introducer 150d may include a weakened region or seam (not shown) to facilitate tearing.

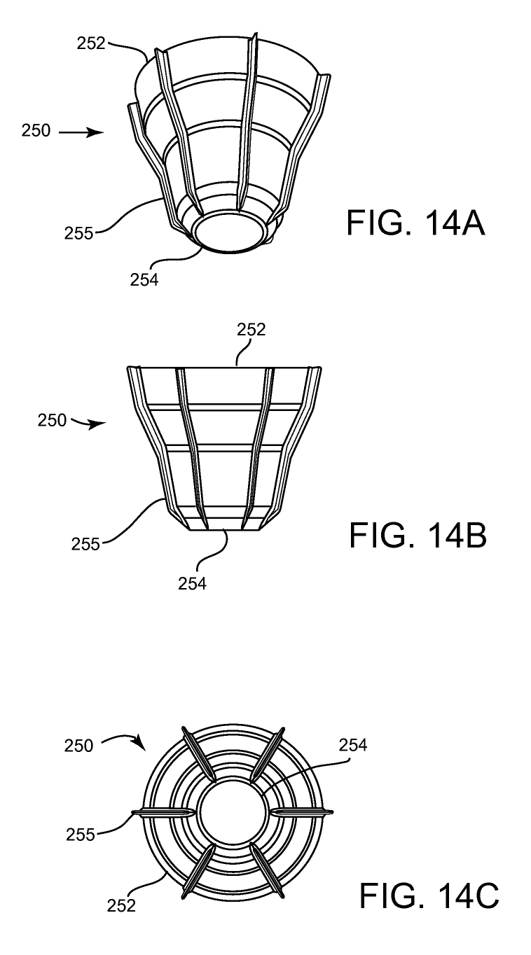

Turning to FIGS. 14A-14C, another embodiment of a valve introducer 250 is shown, which includes a tubular body including proximal end distal ends 252, 254, similar to the previous embodiments. Unlike the previous embodiments, the tubular body includes a plurality of longitudinal pleats or ridges 255 that extend at least partially between the proximal and distal ends 252, 254. The pleats 255 may provide a relatively small amount of flexibility, e.g., to allow the valve introducer 250 to be compressed radially inwardly and/or expanded radially outwardly, if desired. The pleats 255 may have sufficient resilience to bias the valve introducer 250 to return to its relaxed shape when free from external forces.

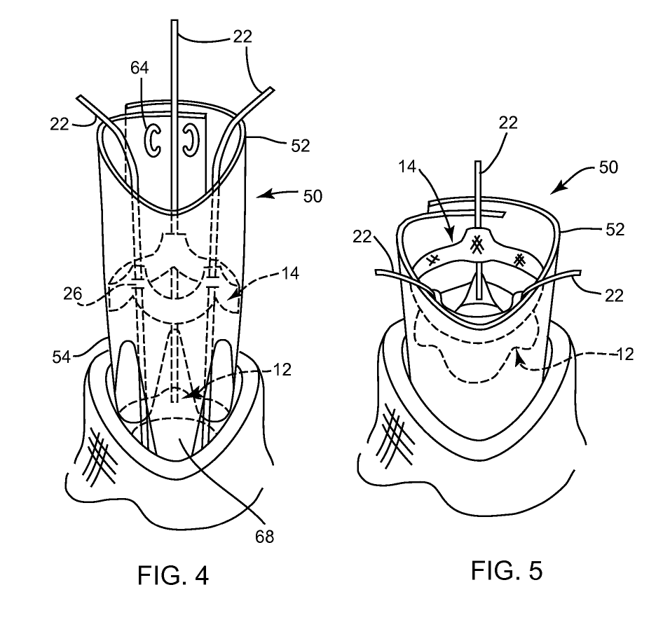

With reference to FIGS. 2-5, a valve introducer 50 (which may include any of the embodiments described herein) may be included in a system or kit 8 for implanting a heart valve assembly 10 within a biological annulus (not shown). Generally, the heart valve assembly 10 includes a gasket member or other annular prosthesis 12 implantable within a biological annulus, a prosthetic valve 14, e.g., including a mechanical or bioprosthetic heart valve, and the valve introducer 50. Optionally, the system or kit 8 may include other components, e.g., a valve holder and/or other tools (not shown). It should be noted that only a frame of the valve prosthesis 14 is shown in FIGS. 2-5, the leaflets being omitted for clarity. FIGS. 15C and 15D show an exemplary embodiment of a bioprosthetic valve 14 including a plurality of tissue leaflets 30 movable on a frame 32. Additional information on valve prostheses 14 and/or gasket members 12 that may be included in the system or kit 8 may be found in co-pending applications Ser. Nos. 10/646,639, filed Aug. 22, 2003, published as 2005/0043760, Ser. No. 10/681,700, filed Oct. 8, 2003, published as 2005/0080454, Ser. No. 10/765,725, filed Jan. 26, 2004, published as 2005/0165479, Ser. No. 11/069,081, filed Feb. 28, 2005, published as 2006/0195184, Ser. No. 11/144,254, filed Jun. 3, 2005, published as 2006/0276888, Ser. No. 11/279,246, filed Apr. 10, 2006, published as 2006/0235508, Ser. No. 11/420,720, filed May 26, 2006, published as 2007/0016285, 60/746,038, filed Apr. 29, 2006, Ser. No. 11/567,735, filed Dec. 6, 2006, and Ser. No. 11/668,459, filed Jan. 29, 2007, the entire disclosures of which are expressly incorporated by reference herein.

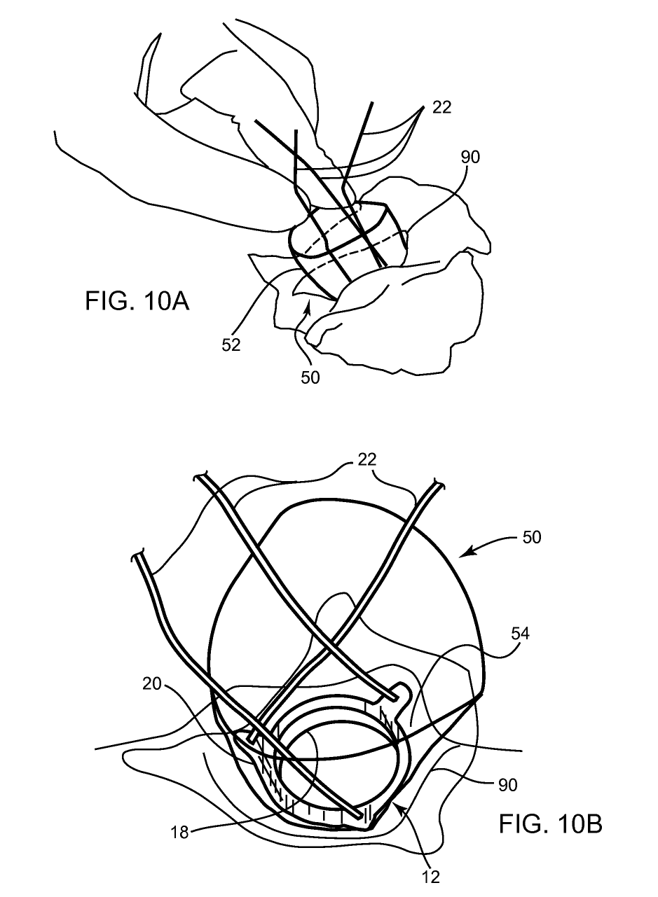

Turning to FIGS. 10A and 10B, during use, the gasket member 12 may be implanted within a patient's body, e.g., within or adjacent to a biological annulus 90. The biological annulus 90 may be the site for replacement of an existing natural or previously implanted heart valve, such as a tricuspid, mitral, aortic, or pulmonary valve within a patient's heart (not shown). As shown in FIGS. 10A, 10B, and 15A-15D, the gasket member 12 may include an annular ring 18, a sewing cuff 20 extending radially outwardly from the annular ring 18, and a plurality of elongate guide rails 22. Optionally, the gasket member 12 may include a collar (not shown) extending upwardly from the annular ring 18 and/or sewing cuff 20, e.g., for receiving the prosthetic valve 14 therein. Optionally, at least a portion of the gasket member 12 may be compressed or otherwise contracted into a relatively small diameter to facilitate advancement into the biological annulus 90, e.g., using a delivery tool (not shown), such as those disclosed in the applications incorporated by reference herein. The gasket member 12 may be at least partially released and/or positioned within the biological annulus 90.

Once properly positioned, a plurality of fasteners, e.g., clips, staples, sutures, and the like (not shown), may be directed through a portion of the gasket member 12, e.g., through the sewing cuff 20, into tissue surrounding the biological annulus 90 to secure the gasket member 12 relative to the biological annulus 90. Additional information regarding the gasket member 12 and methods for delivering and/or securing it are disclosed in the applications incorporated by reference herein.

After securing the gasket member 12 relative to the biological annulus 90, the distal end 54 of the valve introducer 50 may be introduced into the biological annulus 90, e.g., into a sinus cavity above a native aortic valve site being replaced. As shown in FIGS. 10A and 10B, if the gasket member 12 includes guide rails 22, the guide rails 22 may be inserted into the valve introducer 50 such that the valve introducer 50 is introduced into the biological annulus 90 around the guide rails. If the valve introducer 150 includes holes 170, as shown in FIG. 15B adjacent the proximal end 154, the guide rails 22 may be inserted from outside the distal end 54 through the holes 170 into the interior of the valve introducer 150 and out the proximal end 152.

When the valve introducer 40 is fully inserted, the proximal end 52 of the valve introducer 50 may be exposed and the guide rails 22 (if provided) may extend from the proximal end 52 of the valve introducer 50, as shown in FIG. 10A. Optionally, as described above, the valve introducer 50 (or petals or other components thereof) may be compressed radially inwardly to reduce a profile of the distal end 54 and/or to otherwise facilitate introduction of the distal end 54 into the biological annulus, e.g., to facilitate advancement through the aorta or other access location. If compressed, the valve introducer 50 may be released after the distal end 54 is introduced into the biological annulus 90.

After introduction, the distal end 54 of the valve introducer 50 may be disposed adjacent the gasket member 12. Optionally, as shown in FIG. 10B, the distal end 54 may be pressed against the gasket member 12, e.g., to prevent further movement of the gasket member 12 and/or the distal end 54 of the valve introducer 50. In addition, this may prevent a portion of the sewing cuff 20 from rolling or folding inwardly during introduction of the prosthetic valve. If the valve introducer 50 has a multiple sided shape, the valve introducer 50 may be rotated or otherwise positioned to angularly align the distal end relative to the gasket member 12 and/or the biological annulus. For example, the valve introducer 50 may be rotated such that the apices of the sides are aligned with lobes of the sewing cuff 20.

Returning to FIGS. 15B and 15C, if the valve introducer 150 includes a plurality of holes 170 adjacent the distal end 154, guide rails 22 from the gasket member 12 may be inserted through the holes 170, and then the valve introducer 150 may be advanced along the guide rails 22 towards the gasket member 12. Thus, the holes 170 and guide rails 22 may maintain the valve introducer 150 in a desired angular orientation during advancement towards the gasket member 12. Optionally, the guide rails 22 may include detents or other connectors 24 adjacent the gasket member 12, which may be received through the holes 170 when the valve introducer 150 is directed towards the gasket member 12. Once the connectors 24 are received through the holes 170, the connectors 24 may prevent substantial proximal movement of the valve introducer 150 away from the gasket member 12, thereby securing the distal end 154 against, adjacent, within, or otherwise relative to the gasket member 12, as best seen in FIG. 15C.

If the gasket member 12 includes a collar (not shown) the petals 68 may be sized and/or shaped to be received at least partially within the collar. Thus, the petals 68 may facilitate introducing the prosthetic valve 14 into the collar. The collar and/or prosthetic valve 14 may include one or more connectors for securing the prosthetic valve 14 once received in the collar.

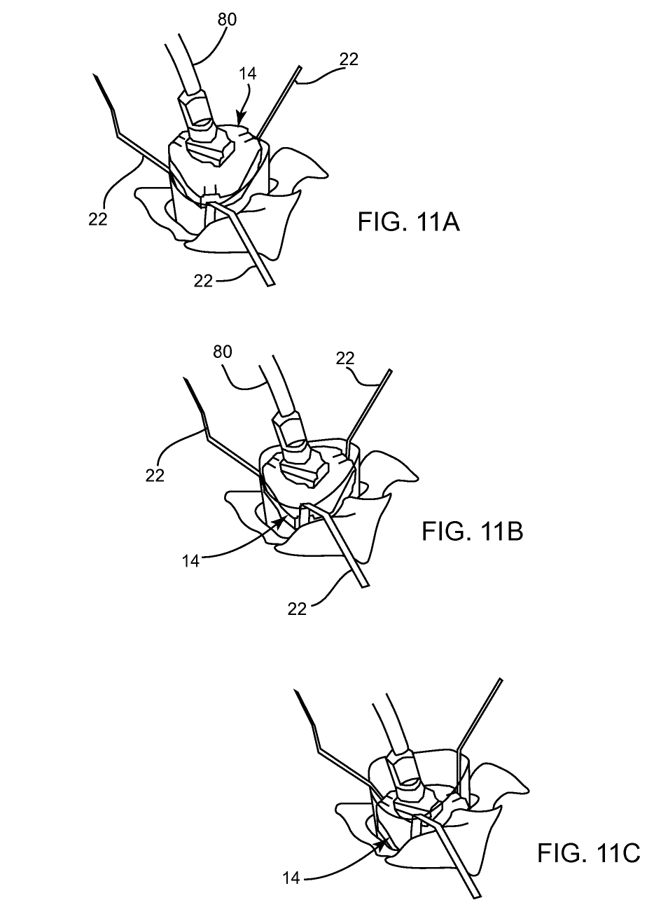

Turning to FIGS. 11A-11C, with the distal end 54 of the valve introducer 50 secured or maintained against or adjacent the gasket member 12, the prosthetic valve 14 may be advanced through the valve introducer 50 into the biological annulus 90, e.g. using a valve holder or other delivery tool 80. In one embodiment, as shown in FIG. 15C, the prosthetic valve 14 may include one or more receptacles 26, e.g., slots in the fabric covering, cans, buckles, and the like corresponding to the guide rails 22 of the gasket member 12. Before inserting the prosthetic valve 14 into the valve introducer 50, the guide rails 22 may be inserted through respective receptacles 26, and then the prosthetic valve 14 may be directed into the proximal end 52 of the valve introducer 50. Thus, the prosthetic valve 14 may be maintained in a predetermine angular orientation as the prosthetic valve 14 is advanced through the valve introducer 50.

If the valve introducer 50 and the valve prosthesis 14 have corresponding similar shapes, the valve prosthesis 14 may be introduced into the valve introducer 50 only after properly aligning the valve prosthesis 14, even if no guide rails are provided. The valve prosthesis 14 may then be advanced through the valve introducer 50 and into contact with the gasket member 12.

Simultaneously with introduction through the valve introducer 50, the valve prosthesis 14 may be advanced along the guide rails 22, thereby further guiding the valve prosthesis 14 towards the gasket member 12. The guide rails 22 and/or valve prosthesis 14 may include connectors (not shown), e.g., as described in the applications incorporated by reference herein, which may automatically engage one another when the prosthetic valve 14 is positioned against the gasket member 12. Alternatively or in addition, the prosthetic valve 14 may be secured to the gasket member 12 using other connectors, e.g., on the prosthetic valve 14 and/or gasket member 12, using sutures (not shown), and the like. For example, if the gasket member 12 includes a collar extending upwardly from the annular ring 18 and/or sewing cuff 20, the collar may include one or more connectors, e.g., detents, drawstring, and the like, which may secure the prosthetic valve 14 within the collar.

Once the prosthetic valve 14 is secured relative to the gasket member 12, the valve introducer 50, valve holder 80, and/or any other tools may be removed, leaving the heart valve assembly 10 behind, e.g., as shown in FIG. 15D. If the valve introducer 50 includes holes receiving the guide rails 22, the valve introducer 50 may be at least partially torn or otherwise separated to allow removal from the guide rails 22. For example, the valve introducer 50 may include weakened regions or seams (not shown) between the holes and the distal end 54, which may tear when the valve introducer 50 is pulled relative to the guide rails 22. When the valve introducer 50 is pulled, the petals 68 or any other portion of the valve introducer 50 between the prosthetic valve 14 and the gasket member 12 (e.g., the collar and/or sewing cuff 20) may be pulled therefrom.

Optionally, the valve introducer 50 may include one or more features allowing the valve introducer 50 to be torn or otherwise separated into one or more sheets, as described previously. This may facilitate removing the valve introducer 50 from around the implanted heart valve assembly 10. For example, tearing a longitudinal seam in the valve introducer 50 or separating overlapping edges may allow the valve introducer 50 to be directed laterally away from the heart valve assembly 10 to facilitate removal.

The guide rails 22 (if provided) may be cut or otherwise severed, e.g., before or after removing the valve introducer 50. The procedure may then be completed using known procedures.

The valve introducers described herein may facilitate advancing a prosthetic valve through a passage communicating with a biological annulus. For example, for aortic valve applications, the valve introducer may facilitate advancing a prosthetic valve past the sino-tubular junction above the sinus of Valsalva. The valve introducer may provide an inexpensive single use device, which may be disposed of after implanting the heart valve assembly. Alternatively, the valve introducer may be resterilized for reuse in a subsequent procedure.

It will be appreciated that elements or components shown with any embodiment herein are exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

We claim:

1. A system for implanting a heart valve assembly within a biological annulus, comprising:
an annular prosthesis implantable within a biological annulus;
a valve prosthesis connectable to the annular prosthesis; and
a valve introducer comprising a tubular body including a proximal end for receiving the valve prosthesis therein, and a distal end sized for introduction into a biological annulus for introducing the valve prosthesis through the valve introducer towards the annular prosthesis after the annular prosthesis is implanted within a biological annulus.

2. The system of claim 1, wherein the gasket member comprises a plurality of elongate elements extending therefrom, and wherein the valve introducer comprises a plurality of holes adjacent the distal end for receiving respective elongate elements therethrough.

3. The system of claim 2, wherein the valve introducer comprises tearable regions adjacent the holes for facilitating separation of the distal end of the valve introducer from the plurality of elongate elements.

4. The system of claim 2, wherein the elongate elements comprise one or more connectors adjacent the gasket member, the connectors receivable through the holes when the valve introducer is advanced adjacent the gasket member to prevent subsequent substantial proximal movement of the valve introducer relative to the gasket member.

5. The system of claim 1, wherein the gasket member comprises a collar or seat for receiving the valve prosthesis, and wherein the distal end of the valve introducer is sized to be received at least partially in the collar or seat.

6. The system of claim 1, wherein the valve introducer is substantially transparent.

7. The system of claim 1, wherein the valve introducer comprises a tearable region extending at least partially between the proximal and distal ends of the valve introducer for separating the valve introducer to facilitate removal.

8. The system of claim 7, wherein the tearable region comprises at least one of a perforated region, a thin-walled region, and a weakened region extending between the proximal and distal ends of the valve introducer.

9. The system of claim 7, wherein the valve introducer further comprises a rip cord for tearing the valve introducer along the tearable region.

10. The system of claim 9, wherein the rip cord is received through a hole adjacent the distal end of the valve introducer.

11. The system of claim 9, wherein the rip cord comprises a loop extending between the proximal and distal ends of the valve introducer.

12. The system of claim 1, wherein at least a portion of the tubular body has a multiple sided cross-section.

13. The system of claim 12, wherein the distal end of the tubular body has a cross-section similar to the valve prosthesis, thereby substantially maintaining the valve prosthesis in a desired angular orientation during introduction from the valve introducer.

14. The system of claim 1, wherein the tubular body is tapered between the proximal and distal ends.

15. The system of claim 1, wherein the distal end is smaller than the proximal end.

16. The system of claim 1, wherein tubular body is movable for reducing a profile of at least the distal end of the tubular body.

17. The system of claim 1, wherein the distal end comprises a plurality of petals that may be manipulated to reduce the profile of the distal end.

18. The system of claim 1, further comprising a pivot point adjacent the proximal end, allowing the valve introducer to be compressed at a location below the pivot point to reduce the profile of the distal end.

19. The system of claim 1, wherein the tubular body comprises a flat sheet of material shaped into the tubular body.

20. The system of claim 19, wherein opposing edges of the flat sheet comprises one or more cooperating connectors that secure the tubular body in a substantially tubular shape.

21. A valve introducer for delivering a valve prosthesis into a biological annulus, comprising a tubular body including a proximal end for receiving a valve prosthesis therein, and a distal end sized for introduction into a biological annulus for guiding the valve prosthesis therein.

22. The valve introducer of claim 21, wherein the gasket member comprises a plurality of elongate elements extending therefrom, and wherein the valve introducer comprises a plurality of holes adjacent the distal end for receiving respective elongate elements therethrough.

23. The valve introducer of claim 22, wherein the valve introducer comprises tearable regions adjacent the holes for facilitating separation of the distal end of the valve introducer from the plurality of elongate elements.

24. The valve introducer of claim 22, wherein the elongate elements comprise one or more connectors adjacent the gasket member, the connectors receivable through the holes when the valve introducer is advanced adjacent the gasket member to prevent subsequent substantial proximal movement of the valve introducer relative to the gasket member.

25. The valve introducer of claim 21, wherein the gasket member comprises a collar or seat for receiving the valve prosthesis, and wherein the distal end of the valve introducer is sized to be received at least partially in the collar or seat.

26. The valve introducer of claim 21, wherein the valve introducer is substantially transparent.

27. The valve introducer of claim 21, wherein the valve introducer comprises a tearable region extending at least partially between the proximal and distal ends of the valve introducer for separating the valve introducer to facilitate removal.

28. The valve introducer of claim 27, wherein the tearable region comprises at least one of a perforated region, a thin-walled region, and a weakened region extending between the proximal and distal ends of the valve introducer.

29. The valve introducer of claim 27, wherein the valve introducer further comprises a rip cord for tearing the valve introducer along the tearable region.

30. The valve introducer of claim 29, wherein the rip cord is received through a hole adjacent the distal end of the valve introducer.

31. The valve introducer of claim 29, wherein the rip cord comprises a loop extending between the proximal and distal ends of the valve introducer.

32. The valve introducer of claim 21, wherein the at least a portion of the tubular body has multiple sided cross-section.

33. The valve introducer of claim 22, wherein the distal end of the tubular body has a cross-section similar to the valve prosthesis, thereby substantially maintaining the valve prosthesis in a desired angular orientation during introduction from the valve introducer.

34. The valve introducer of claim 21, wherein the tubular body is tapered between the proximal and distal ends.

35. The valve introducer of claim 21, wherein the distal end is smaller than the proximal end.

36. The valve introducer of claim 21, wherein the tubular body is movable for reducing a profile of the distal end.

37. The valve introducer of claim 21, wherein the distal end comprises a plurality of petals that may be manipulated to reduce the profile of the distal end.

38. The valve introducer of claim 21, further comprising a pivot point adjacent the proximal end, allowing the valve introducer to be compressed at a location below the pivot point to reduce the profile of the distal end.

39. The valve introducer of claim 21, wherein the tubular body comprises a flat sheet of material shaped into the tubular body.

40. The valve introducer of claim 39, wherein opposing edges of the flat sheet comprises one or more cooperating connectors that secure the tubular body in a substantially tubular shape.

41. A method for implanting a prosthetic heart valve assembly to replace a natural or prosthetic heart valve implanted within a biological annulus below a passage, comprising:
introducing an annular member into the biological annulus;
securing the annular member relative to the biological annulus;
introducing a distal end of a valve introducer into the biological annulus;
advancing a valve prosthesis through the valve introducer towards the annular member; and
securing the valve prosthesis relative to the annular member within the biological annulus.

42. The method of claim 41, wherein the valve introducer and valve prosthesis have similar predetermined shapes, and wherein the valve prosthesis is oriented to align the predetermined shapes before advancing the valve prosthesis through the valve introducer.

43. The method of claim 41, further comprising compressing or manipulating the valve introducer to reduce a profile of the distal end before or during introduction into the passage.

44. The method of claim 41, wherein the distal end of the valve introducer is pressed against the annular member before advancing the valve prosthesis through the valve introducer.

45. The method of claim 41, wherein the gasket member comprises a plurality of elongate elements extending therefrom, the method further comprising inserting the elongate elements through respective holes in the distal end of the valve introducer before introducing the distal end of the valve introducer into the biological annulus.

46. The method of claim 45, further comprising removing the valve introducer from the biological annulus, thereby tearing the valve introducer adjacent the holes to separate the distal end of the valve introducer from the elongate elements.

47. The method of claim 45, wherein the distal end of the valve introducer is introduced into the biological annulus until the distal end of the valve introducer is disposed adjacent the gasket member.

48. The method of claim 47, wherein one or more connectors on the elongate members are received through the respective holes when the valve introducer is disposed adjacent the gasket member, thereby substantially securing the distal end of the valve introducer relative to the gasket member.

49. The method of claim 41, wherein the gasket member comprises a collar or seat for receiving the valve prosthesis, and wherein the distal end of the valve introducer is received at least partially in the collar or seat when the distal end of the valve introducer is introduced into the biological annulus.

50. The method of claim 41, wherein tissue surrounding the biological annulus is monitored through a wall of the valve introducer as the distal end of the valve introducer is introduced into the biological annulus.

51. The method of claim 41, further comprising removing the valve introducer from the biological annulus after securing the valve prosthesis relative to the annular member within the biological annulus.

52. The method of claim 51, wherein removing the valve introducer from the biological annulus comprises separating the valve introducer into one or more sheets.

53. The method of claim 52, wherein separating the valve introducer comprises a tearing a tearable region extending at least partially between the proximal and distal ends of the valve introducer.

54. The method of claim 52, wherein separating the valve introducer comprises pulling a rip cord to tear at least a portion of the valve introducer.
